(19) United States
(12) Patent Application Publication
(10) Pub. No.: US 2009/0099863 A1
(43) Pub. Date: Apr. 16, 2009

(54) Rehabilitation therapy camp

(75) Inventors: Omer Einav, Emek Hefer (IL); Richard M. Mahoney, Westmont, NJ (US); Haim Einav, Tel-Aviv (IL); Ester Zohar, Givataim (IL)

Correspondence Address: MARTIN D. MOYNIHAN d/b/a PRTSI, INC., P.O. BOX 16446, ARLINGTON, VA, 22215, US

(73) Assignee: Motorika Limited, Hamilton (BM)

(21) Appl. No.: 11/907,511

(22) Filed: Oct. 12, 2007

(51) Int. Cl. A61B 5/00

(52) U.S. Cl. 705/2

(57) ABSTRACT

A method of providing rehabilitation modalities to a plurality of patients in a multipurpose location for a fixed period of time, the method comprises designating a first plurality of patients to attend rehabilitation sessions in a first multipurpose location for a first fixed period of time, providing a plurality of rehabilitation modalities at the first multipurpose location for the first fixed period of time, and allocating the first plurality of patients at the first multipurpose location to multiple rehabilitation sessions on one or more of the plurality of rehabilitation modalities.

### FIELD AND BACKGROUND OF THE INVENTION

The present invention relates to locating specialized rehabilitation modalities in a multipurpose location for a fixed period.

A stroke creates neurological deficits that affect, inter alia, speech, cognition, memory, and motor control. Following a stroke, extensive rehabilitation that addresses each neurological deficit should be immediately begun to prevent any residual functions from being lost and/or inappropriate compensations replacing the functions resulting from the neurological deficit.

Additionally, post-stroke patients may often have special long-term needs that require low intensity rehabilitation to prevent neurological relapse as much as two years, or even three years, post stroke.

Unfortunately, post-stroke patients are often unable to follow the necessary rehabilitation regimens due to a variety of difficulties. For example, a patient may experience transportation problems to reach a first clinic equipped with speech therapy modalities. Additionally, a second clinic equipped with motor-skill rehabilitation modalities may be located far from the first clinic, creating further transportation difficulties. Moreover, the first and second clinics may both be open during concurrent, and short, daily periods that preclude scheduling two crucial therapy sessions on the required daily basis.

As a result of the above-noted difficulties, post stroke patients may be required to use one or more home-based therapy modality units administered by a therapist who is inexperienced in assessing patient progress and fails to progress the patient through a required, increasingly complex, rehabilitation regimen. As a result, the few weekly therapy sessions with the therapist, even with intermittent therapy sessions in a therapy clinic, will woefully fail in effectively reversing stroke-related neurological deficits.

There is thus a widely recognized need for providing acute and long-term therapy on specialized therapy modalities to post-stroke patients, devoid of the above-noted limitations.

### SUMMARY OF THE INVENTION

According to one aspect of the present invention, there is provided a method of providing rehabilitation modalities to a plurality of patients in a multipurpose location for a fixed period, the method comprising designating a first plurality of patients to attend rehabilitation sessions in a first multipurpose location for a first fixed period of time, providing a plurality of rehabilitation modalities at the first multipurpose location for the first fixed period of time, and allocating the first plurality of patients at the first multipurpose location to multiple rehabilitation sessions on one or more of the plurality of rehabilitation modalities.

In embodiments, the method includes removing the plurality of rehabilitation modalities from the first multipurpose location following the first fixed period of time.

In embodiments, the method includes repeating the procedures in a second multipurpose location with a second group of a plurality of patients for second fixed period of time.

In embodiments, at least one of the plurality of rehabilitation modalities is utilized in the one first multipurpose location, and in the one second multipurpose location.

In embodiments, additional services are provided in the one first multipurpose location, and in the one second multipurpose location; including at least one service related to at least one of Physical Medicine diagnostic equipment and Physical Medicine treatment equipment.

In embodiments, the method further includes collecting data on the ongoing progress of the first plurality of patients at the first multipurpose location and the second plurality of patients at the second multipurpose location, evaluating the collected data, and providing statistical predictions for at least one of optimal therapy regimens and patient recovery time for a given condition.

In embodiments, the first period of time and the second period of time are at least between about one week and about six weeks.

In embodiments, the first period of time and the second period of time are no more than between about seven weeks and about twenty weeks.

According to another aspect of the present invention, there is provided a method of allocating rehabilitation modalities for treating a plurality of patients for a fixed period of time, the method comprising, designating a first group comprising a plurality of patients having similar conditions requiring similar rehabilitation modalities, scheduling the first group of the plurality of patients having similar conditions to attend rehabilitation sessions in a first multipurpose location for a first fixed period of time, and allocating a plurality of rehabilitation modalities appropriate for the first group of a plurality of patients having similar conditions at the first multipurpose location for the first fixed period of time.

In embodiments, the method includes removing the plurality of rehabilitation modalities from the first multipurpose location following the first fixed period of time.

In embodiments, the method includes repeating the procedures in a second multipurpose location with a second group of a plurality of patients for a second fixed period of time.

In embodiments, the first group of patients and the second group of patients are determined based upon similar conditions of age, illness, illness onset date, severity of symptoms, and recovery history.

In further embodiments, the first group of patients and the second group of patients are determined by Physical Medicine treatment needs.

In further embodiments, the first group of patients and the second group of patients are determined by neurological indications from the group of neurological indications comprising stroke, cerebral palsy, Charcot-Marie-Tooth (CMT), amyotrophic lateral sclerosis (ALS), brain injury, multiple sclerosis, Parkinson's disease, and/or spinal cord injury.

According to still another aspect of the present invention, there is provided a method of locating a plurality of rehabilitation modalities in a multipurpose location for a fixed period of time, the method comprising, designating a first group comprising a plurality of patients having similar rehabilitation needs to attend rehabilitation sessions in a first multipurpose location for a first fixed period of time, and delivering a plurality of rehabilitation modalities required for the similar rehabilitation needs to the first multipurpose location for the first fixed period of time.

In embodiments, the method includes removing the plurality of rehabilitation modalities from the first multipurpose location following the first fixed period of time.

In embodiments, the method includes repeating the procedures in a second multipurpose location with a second group of a plurality of patients having similar rehabilitation needs for a second fixed period of time.

In embodiments, at least a portion of the plurality of rehabilitation modalities includes a programmable robotic component.

In embodiments, the programmable robotic component includes an automatic positional adjustment configured to automatically adjust position for each patient based upon entry of a patient identifier.

In embodiments, the programmable robotic component includes an automatic height adjustment configured to automatically adjust height of the modality component for each patient based upon entry of a patient identifier.

In embodiments, the programmable robotic component includes a therapy progress-evaluating module configured to evaluate patient progress during a given session and provide, on the given modality during the session, at least one of: increased therapeutic challenge on the modality and decreased therapeutic challenge on the modality.

In embodiments, the method includes allocating services to the first multiple multipurpose location and the second multiple multipurpose location from the group of services comprising services related to therapists, Physical Medicine diagnostic equipment, Physical Medicine treatment equipment, neurological treatment units, computer systems, billing modules, communication networks and patient transportation.

According to a further aspect of the present invention, there is provided a method of locating a multiple multipurpose location for treating a group of patients for a fixed period of time, the method comprising, locating a first multipurpose location, designating a first group of a plurality of patients located within a predetermined radius of the multipurpose location, providing a plurality of rehabilitation modalities in the first multipurpose location for a first fixed period of time, and treating the first group of a plurality of patients with the plurality of rehabilitation modalities for the first fixed period of time.

In embodiments, the method includes removing the plurality of rehabilitation modalities from the multipurpose location following the first fixed period of time.

In embodiments, the method includes repeating the procedures in a second multipurpose location with a second group of a plurality of patients for a second fixed period of time.

The present invention successfully addresses the shortcomings of the presently known configurations by providing one or more temporary rehabilitation therapy camps.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method. The term “method” refers to manners, means, techniques and procedures for accomplishing a given task including, but not limited to, those manners, means, techniques and procedures either known to, or readily developed from known manners, means, techniques and procedures by practitioners of the therapeutic treatment.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of embodiments of the invention could be implemented as a chip or a circuit. As software, selected steps of embodiments of the invention could be implemented as a plurality of software instructions being executed by a computer using any suitable operating system. In any case, selected steps of the method and system of embodiments of the invention could be described as being performed by a data processor, such as a computing platform for executing a plurality of instructions.

### BRIEF DESCRIPTION OF THE DRAWINGS

Embodiments of the invention is herein described, relating to methods and devices for establishing temporary rehabilitation therapy camps, is by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of embodiments of the invention in more detail than is necessary for a fundamental understanding of the invention, the description taken with the drawings making apparent to those skilled in the art how the several forms of the invention may be embodied in practice.

In the drawings:

FIG. 1 shows a flow chart for planning and executing a therapy camp, according to embodiments of the invention; and

FIG. 2 shows typical modalities of therapy and set up of a therapy camp, according to embodiments of the invention.

### DESCRIPTION OF THE PREFERRED EMBODIMENTS

Embodiments of the present invention relate to methods and therapy modalities for establishing temporary rehabilitation therapy camps that can be set up in a plurality of locations, each location providing quality therapy modalities to patients for a fixed period of time. The principles and operation of each camp according to embodiments of the present invention may be better understood with reference to the drawings and accompanying descriptions.

Before explaining at least one embodiment of the invention in detail, it is to be understood that embodiments of the invention are not limited in application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Referring now to the drawings:

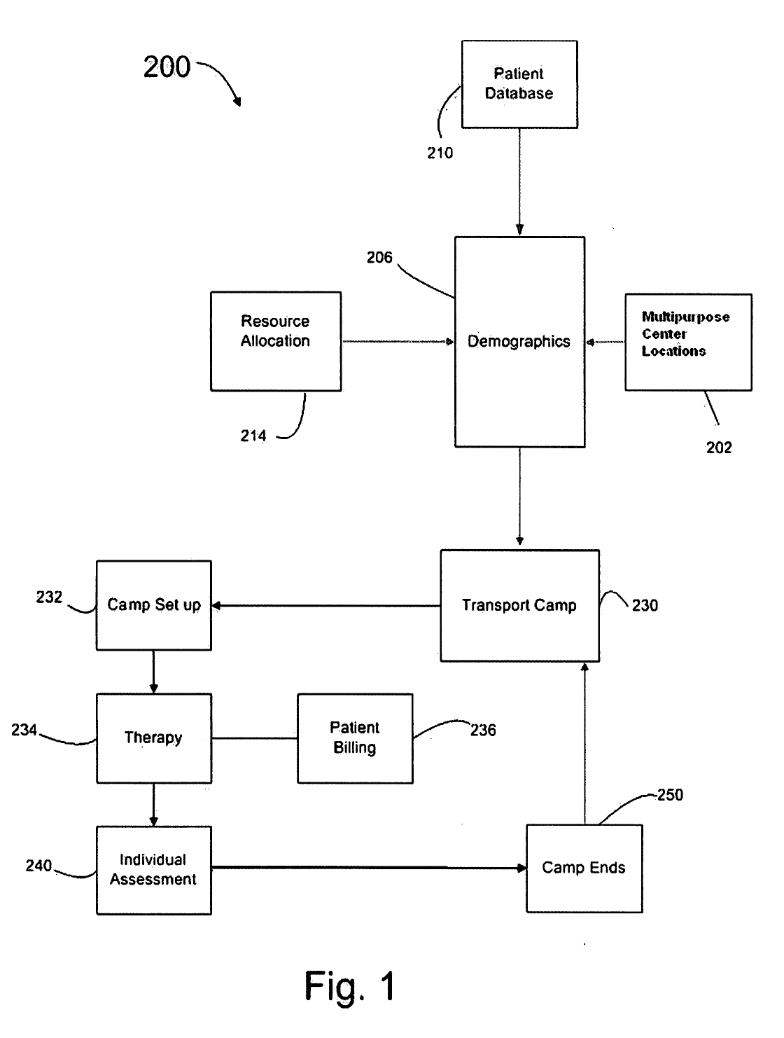

FIG. 1 shows a flow chart 200 of a therapy camp preparation and function wherein at a patient database stage 210, patient data is entered. Patient data includes, inter alia, profiles on patient age, height, neurological deficit, and medical history.

Groups of patients are formed so that patients having similar post-stroke neurological therapy needs are in a given group. For example patients exhibiting upper extremity motor control deficit, all of whom require upper extremity motor coordination and strengthening therapy modalities, are grouped together. Additional therapy groups may include patients with lower extremity motor coordination deficit, cognitive deficit, and/or speech pathology.

As used herein, the terms “neurological conditions” and “neurological indications” refer not only to post-stroke treatment, but also any one of a variety of neurological conditions and/or indications, including, inter alia, cerebral palsy, Charcot-Marie-Tooth (CMT), amyotrophic lateral sclerosis (ALS), brain injury, multiple sclerosis, Parkinson's disease, and/or spinal cord injury.

Consequently, in still further embodiments, groups of patients are formed so that patients having similar neurological indications are in a given group.

For example, multiple groups are optionally formed, with each group dedicated to another neurological indication, for example stroke, cerebral palsy, Charcot-Marie-Tooth (CMT), amyotrophic lateral sclerosis (ALS), brain injury, multiple sclerosis, Parkinson's disease, and/or spinal cord injury.

In still other embodiments, groups are further divided according to Physical Medicine needs. For example, post-stroke patients exhibiting diabetes and associated disorders comprising diabetic neuropathy and/or renal insufficiency are grouped together.

Further, personnel and equipment allocation, noted below, are determined based upon the groups formed, whether according to post-stroke similar neurological needs, physical medicine needs and/or neurological indication needs.

The groups are now further categorized into subgroups at a demographics stage 206, according to geographic areas where the patients are located. For example, an upper motor control therapy group is divided into two subgroups: a first subgroup in city A and a second subgroup in city B.

At a multipurpose center location database stage 202 a first multipurpose center located in city A is reserved for between about one and twenty consecutive weeks of therapy, and a second multipurpose center located in city B is reserved for between about one and twenty consecutive weeks of therapy thereafter.

Based upon the location of the multipurpose center and modality needs of the subgroups to receive therapy at each location, a resource allocation stage 214 is accessed to schedule resource transportation by a van or truck. Resources include, inter alia, Physical Medicine diagnostic equipment, Physical Medicine treatment equipment, neurological therapy equipment, computer systems, and communication systems

Each city is additionally allocated therapists, Physical Medicine diagnosticians, and/or Medical Doctors that are trained in treating the various subgroups. Therapists are notified of their individual schedules and, if necessary, are booked into local hotels if the location is distant from their home.

Additionally, Medical personnel employment agencies are optionally contracted to supply therapists, Physical Medicine diagnosticians, and/or Medical Doctors, particularly during peak hours of patient visits and resource utilization.

At a transport camp stage 230, portable specialized therapy modalities for treating the various subgroups are packed and loaded into a van and transported to city A. At a camp setup stage 232, personnel associated with the multipurpose center located in city A, and/or personnel associated with the therapy camp, unpack and move the equipment into the multipurpose center.

Therapy camp in city A opens after being fully equipped with therapy equipment, computer systems and communication systems that facilitate running the therapy camp in an efficient manner, as described in U.S. application Ser. No. 11/348,128 entitled “Rehabilitation Methods” filed 6 Feb. 2006, now published as 20060277074, the disclosure of which is hereby fully incorporated herein by reference.

Therapy is administered at a therapy stage 234, and at a billing stage 236, bills for therapy services are sent to patients and/or third party payers as described below.

During the camp session, at an individual assessment stage 240, each member of a given subgroup is assessed and it is determined whether to maintain current therapy regimen or to change the therapy regimen.

After a fixed period of time, at a camp end stage 250, all equipment is packed onto the van or truck and, during transport camp stage 230, the therapy camp is transported from multipurpose center located in city A to multipurpose center located in city B.

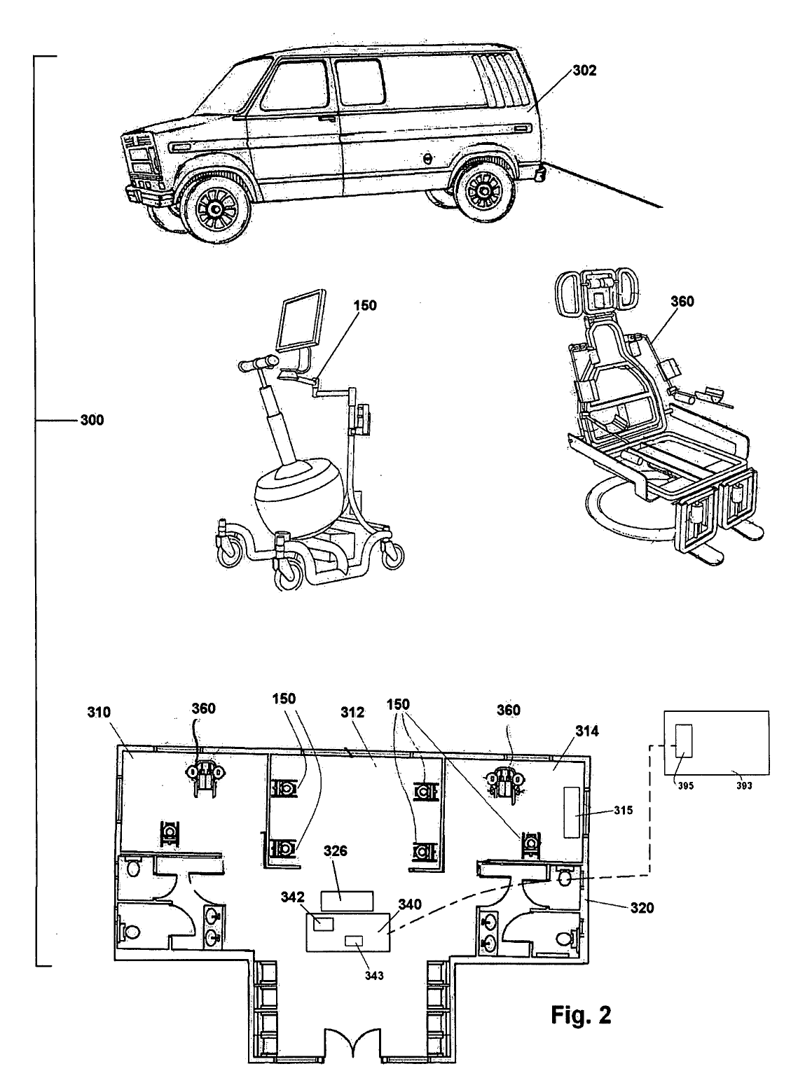

FIG. 2 shows the set up of a therapy camp 300 in which portable therapy modalities, including a chair-based modality 360 and a mobile cart modality 150, as described in US Patent application publication number 20060277074 noted above, are unloaded from a truck or a van 302 and located in a multipurpose facility 320 where treatments are provided.

The phrase “in multipurpose facility” with respect to therapy treatments on modalities 360 and/or 150, is defined herein as providing therapy treatments on modalities 360 and/or 150 “within” the multipurpose facility.

As noted above, modalities 360 and 150 have been chosen based upon the neurological therapy and/or physical medicine needs of patients to be treated. Further, the number of pieces of equipment to be unloaded from truck or van 302 has been determined according to the volume of the patients in each subgroup and the size and layout of multipurpose facility 320.

Multipurpose facility 320 has, in a non-limiting example, three treatment rooms 310, 312 and 314 that are equipped with modalities 360 and/or 150 in a manner that optimizes patient flow as described below.

During the period of therapy camp 300 in city A, patients attend frequent intensive therapy sessions on a daily basis that maximize recovery from strokes and other neurological conditions and indications. Alternatively or additionally, at least one of treatment rooms 310, 312 and 314 are equipped with Physical Medicine diagnostic and/or treatment equipment 315 comprising, for example an EGK unit, treadmill and/or portable x-ray.

As described in US Patent application publication number 20060277074 noted above, in addition to patient history information noted above, software modules 342 on a computer system 340 include indication-specific data for each patient that aid in setting therapy regimens for each patient.

Indication-specific data includes, inter alia, temporal locations of non-functional tissue within the brain; date since stroke occurrence, quality of care post stroke, and therapy modalities and progress to date.

In embodiments, at an initial therapy session the therapist enters a patient smartcard into a smartcard reader on computer system 340 and then adjusts the position and/or configuration of therapy modality 150, or modality 360, to the specific patient.

Modality position and/or configuration information can be automatically stored on software modules 342 along with the patient identification code from the smartcard. Each time the patient comes to modality 150 or 360, the smartcard is entered into a smartcard reader on computer system 340 and software modules 342 automatically send digital instructions to robotic components and transducers may adjust configuration and/or position on modality 150 or 360 to the preprogrammed needs for a given patient.

Alternatively, modalities 150 and 360 include individual smartcard readers and a data chip. Upon entering the patient smartcard, the data chip sends digital instructions so robotic components on modality 150 and/or modality 360 automatically adjust configuration and/or position for a given patient.

During the initial therapy session, computer system 340 assesses the patient neurological deficits. Alternatively or additionally, patient neurological deficits are assessed by a therapist using independent assessment regimens, for example Fugl-Meyer assessments and/or modality-based measurement tools that predict Fugl-Meyer assessments.

With these assessments, in addition to patient history data, each time the smartcard is entered, modality 150 or modality 360 provides a specific range of exercises, at a specific speed of movement and starting pattern for the waiting patient.

In existing rehabilitation plans a patient having a given neurological indication may receive multiple therapy modalities, some at a first location and others at a second location; with substantially inadequate communication between the two locations.

The first location may have the patient continually repeat low intensity therapy regimens while at the second location, the patient performs high intensity therapy. As a result, therapy at the first location may fail to adequately help the patient overcome neurological deficit.

In embodiments of the present invention, it has been discovered that therapy camp 300, equipped with multiple therapy modalities 150 and 360; and computer system 340 that continually evaluates patient progress; substantially overcomes the above-noted problems associated with inadequate communication.

In addition, in existing rehabilitation plans, the emphasis is often on quantitative therapy. For example, a patient having a lower extremity neurological deficiency may mount a treadmill set at a 10-degree incline and walk every day for a twenty-minute period. Without continued feedback on the improvement of lower extremity strength, the facility will fail to take the opportunity to increase the degrees of incline to continually challenge and strengthen the lower extremity muscles.

In embodiments of the present invention, it has been discovered that by utilizing continued evaluation on computer system 340, modalities 150 and 360 can be configured in real time to increase therapeutic challenges based upon patient progress during each session; thereby substantially eliminating the above-noted quantitative deficiencies.

In non-limiting embodiments, software modules 342 optionally determine therapy milestones and optionally recommend modification of therapy regimens presented by modalities 150 and 360 thereby providing increased therapeutic challenges to each patient at each session. This continually upgrades patient progress throughout the period of therapy at camp 300 in city A.

Further, computer system 340 assesses patient progress at each session and may change therapy regimens accordingly. For example, a patient who reaches a certain milestone in neurological progress at an earlier-than-anticipated date on a given therapy modality, is automatically provided with an increased therapy pace and/or greater complexity of therapy activity on the given therapy modality.

Additionally, modalities 150 and 360 automatically provide information input into software modules 342 in real time during a given therapy session with respect to patient range of motion, movement smoothness, and resistance score so that factors such as patient fatigue can be assessed.

In the event of patient fatigue, modalities 150 and/or 360 receive digital instructions from computer system 340 during a given session to reduce pull resistance, for example, to allow the patient to regain stamina.

Following regaining of patient stamina, as measured through input from modalities 150 or 360, computer system 340 sends digital instructions to gradually increase the level of pull resistance to the target level set for the session.

Moreover, if computer system 340 detects that a patient using modality 150 requires a rest period prior to beginning modality 360, computer system 340 notifies the therapist of the required rest period via a modality-based audiovisual system and/or a display 326. Additionally, while the fatigued patient rests, computer system 340 directs the therapists to administer therapy to an alternative patient on modality 360, thereby efficiently allocating the use of therapy modality 360.

In embodiments, computer system 340 additionally has the ability to, upon entering the smart card, display patient progress on display 326 so that the therapist and/or a therapy supervisor of therapy camp 300 are apprised of the current patient therapy level and/or progress during therapy camp 300. In this manner, the therapy supervisor can suggest that the therapist change therapy regimens or directly program computer system 340 to change therapy regimens.

However, the information taken by computer system 340 from modalities 150 and 360 generally removes much of the burden of assessing patient progress from the therapist and/or camp therapy supervisor. In this manner, therapists may be employed that are less expert in patient assessment; and therapy camp 300 can employ therapists trained at a lower level and/or fewer supervising therapists, leading to more efficient operation of therapy camp 300.

Further, each therapist can dedicate the entirety of each session to overcoming patient neurological deficits without stopping to perform tests that confirm patient progress.

In embodiments, computer system 340 is linked to the Internet and patients and/or caregivers have the option of accessing a website dedicated to therapy camp 300, enter the patient identification code and download information on patient progress. Such information is particularly valuable to a therapist, for example, that begins working with the patient following the closure of therapy camp 300.

In addition, via the website of therapy camp 300, computer system 340 provides information on the location and types of neurological deficits to be treated at future camp locations.

For example, additional therapy camps 300 may include modalities and/or therapists dedicated to providing non-intensive maintenance treatments for patients who are several months to several years post stroke. The many configurations and therapy plans for additional therapy camps 300 are well known to those familiar with the art.

Each patient, or caregiver, has the option to enter a personal code and access information regarding eligibility for attending one or more of the future therapy camps.

In addition, computer system 340 optionally collects data on the ongoing progress of multiple patients having similar conditions in multiple camps, evaluates the data and provides statistical predictions for optimal therapy regimens and/or patient recovery time for a given condition.

In addition to the above, additional clinical optimization features maximize the efficiency of therapy camp 300, as described in US Patent application publication number 20060277074 noted above.

For example, computer system 340 automatically tracks patient attendance and, should a patient be late or absent, computer system 340 automatically directs a therapist to put another patient on modality 150 or 360.

In embodiments, computer system 340 additionally provides updates in real time so that the therapy supervisor can immediately contact and reschedule patients who miss appointments.

Additionally, the special layout of therapy camp 300 is programmed into computer system 340 so that a patient who uses modality 150 followed by modality 360 can be treated in room 310 or room 314, where both modalities 150 and 360 are located, thereby reducing the time required for the patient to walk between one modality and the next.

At the beginning of therapy camp 300, computer system 340 optionally includes a billing module 343 that optionally produces an estimate of charges for an entire camp period, paid by the patient prior to beginning therapy camp 300. In addition, billing module 343 provides information on remaining patient credit throughout the sessions at therapy camp 300.

Alternatively, computer system 340 is linked to a central computer 395 at a remote station 393 where scheduling of therapy camps occurs. Billing module 343 automatically records patient usage of modules 150 and 360 and sends the information to central computer 395. Thereafter, bills are sent from remote station 393 to third party payers for treatment services. In embodiments, at remote station 393, payment is recorded and receipts and/or bills are sent to the patient and/or caregiver.

In further embodiments, computer 340 provides a printout to each patient upon request at a given session based upon data from central computer 395. The printout optionally includes, inter alia, services that have been paid by the third party payer and services, and/or deductibles that have not been paid by the third party payer and a running total required from the patient.

Payments are optionally received and recorded at remote station 393 so that following closure of therapy camp 300 billing and receipt of payment continues smoothly.

In still further embodiments, central computer 395 monitors activities at the various sites and establishes general standards for maintaining efficiency of each therapeutic camp 300.

The general efficiency standard optionally includes parameters relating to, inter alia, usage of modalities 150 and 360, patient progress, staff supervision at each of modalities 150 and 360, third party payer reimbursement, and operating hours of camp 300.

Central computer 395 continually receives data relating to the general efficiency standards. Should the transferred data indicate that therapy camp 300 is operating below any one of the parameters of the general efficiency standards; central computer 395 optionally issues a report that indicates the deficiencies.

In some instances, for example where a specific third party payer has been withholding payment, a supervisor at remote station 393 initiates an inquiry to the specific third party payer with the goal of receiving payment in a more timely fashion.

In instances where there is inefficient utilization of therapy modalities 150 and 360, a supervisor from remote station 393 may reviews the report with members of the staff on location of therapy camp 300.

It may be found, for example, that changing the layout of therapy modalities 150 and 360 and/or physical medicine equipment 315 in treatment rooms 310, 312 and 314, will sufficiently increase the utilization efficiency of therapy modalities 150 and 360 and/or physical medicine equipment 315.

It is expected that during the life of this patent many relevant therapy modalities and therapy regimens will be developed and the scope of the terms “therapy modalities” and “therapy regimens” are intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art.

Accordingly, it is intended to embrace all such alternatives, modifications and variations that fall within the spirit and broad scope of the appended claims. All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

It is expected that during the life of this patent many relevant physical therapy modalities will be developed and the scope of the term physical therapy modalities and portable modalities. It is appreciated that the invention is intended to include all such new therapy modality technologies a priori.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims. All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of providing rehabilitation modalities to a plurality of patients in a multipurpose location for a fixed period, the method comprising:
a) designating a first plurality of patients to attend rehabilitation sessions in a first multipurpose location for a first fixed period of time;
b) providing a plurality of rehabilitation modalities at said first multipurpose location for said first fixed period of time; and
c) allocating said first plurality of patients at said first multipurpose location to multiple rehabilitation sessions on one or more of said plurality of rehabilitation modalities.

2. The method according to claim 1, wherein at least a portion of said plurality of rehabilitation modalities at said first multipurpose location include a programmable robotic component.

3. The method according to claim 1, wherein said first plurality of patients have similar rehabilitation needs.

4. The method according to claim 1, including:
d) removing said plurality of rehabilitation modalities from said first multipurpose location following said first fixed period of time.

5. The method according to claim 4, including:
e) repeating steps a through d in a second multipurpose location with a second plurality of patients for second fixed period of time.

6. The method according to claim 5, wherein said second plurality of patients have similar rehabilitation needs.

7. The method according to claim 5, wherein at least one of said plurality of rehabilitation modalities is utilized:
in said one first multipurpose location; and
in said one second multipurpose location.

8. The method according to claim 5, wherein additional services are provided in said one first multipurpose location, and in said one second multipurpose location; including at least one service related to at least one of:
Physical Medicine diagnostic equipment; and
Physical Medicine treatment equipment.

9. The method according to claim 5, including:
f) collecting data on an ongoing basis of said first plurality of patients at said first multipurpose location and said second plurality of patients at said second multipurpose location;
g) evaluating said collected data; and
h) providing statistical predictions for at least one of:
optimal therapy regimens; and
patient recovery time for a given condition.

10. The method according to claim 5, wherein said first period of time and said second period of time are at least between about one week and about six weeks.

11. The method according to claim 5, wherein said first period of time and said second period of time are no more than between about seven weeks and about twenty weeks.

12. A method of allocating rehabilitation modalities for treating a plurality of patients for a fixed period of time, the method comprising:
a) designating a first group comprising a plurality of patients having first similar conditions requiring similar rehabilitation modalities;
b) scheduling said first group of said plurality of patients having said first similar conditions to attend rehabilitation sessions in a first multipurpose location for a first fixed period of time; and
c) allocating a plurality of rehabilitation modalities appropriate for said first group comprising said plurality of patients having said first similar conditions in said first multipurpose location for said first fixed period of time.

13. The method according to claim 12, wherein at least a portion of said plurality of rehabilitation modalities include a programmable robotic component.

14. The method according to claim 12, including:
d) removing said plurality of rehabilitation modalities from said first multipurpose location following said first fixed period of time.

15. The method according to claim 14, including:
e) repeating steps a through d in a second multipurpose location with a second group comprising a plurality of patients having second similar conditions for a second fixed period of time.

16. The method according to claim 15, wherein said first group of patients and said second group of patients are determined, based upon similar conditions of:
age;
illness;
illness onset date;
severity of symptoms; and
recovery history.

17. The method according to claim 16, wherein said first group of patients and said second group of patients are determined by Physical Medicine treatment needs.

18. The method according to claim 15, wherein said first group of patients and said second group of patients are determined, based upon similar neurological indications from the group of medical neurological comprising stroke, cerebral palsy, Charcot-Marie-Tooth (CMT), amyotrophic lateral sclerosis (ALS), brain injury, multiple sclerosis, Parkinson's disease, and/or spinal cord injury.

19. A method of locating a plurality of rehabilitation modalities in a multipurpose location for a fixed period of time, the method comprising:
a) designating a first group comprising a plurality of patients having similar rehabilitation needs to attend rehabilitation sessions in a first multipurpose location for a first fixed period of time; and
b) delivering a plurality of rehabilitation modalities required for said first group of said plurality of patients having said similar rehabilitation needs to said first multipurpose location for said first fixed period of time.

20. The method according to claim 19, wherein at least a portion of said plurality of rehabilitation modalities include a programmable robotic component.

21. The method according to claim 19, including:
c) removing said plurality of rehabilitation modalities from said first multipurpose location following said first fixed period of time.

22. The method according to claim 21, including:
d) repeating steps a through c in a second multipurpose location with a second group comprising a plurality of patients having similar rehabilitation needs for a second fixed period of time.

23. The method according to claim 19, wherein at least a portion of said plurality of rehabilitation modalities include a programmable robotic component.

24. The method according to claim 23, wherein said programmable robotic component includes an automatic configuration wherein transducers automatically adjust configuration for each patient on said portion of said plurality of rehabilitation modalities, based upon entry of a patient identifier.

25. The method according to claim 23, wherein said programmable robotic component includes an automatic therapy regimen adjusting transducer configured to automatically adjust a therapy regimen on said portion of said plurality of rehabilitation modalities for each patient based upon entry of a patient identifier.

26. The method according to claim 23, wherein said programmable robotic component includes a therapy progress-evaluating module configured to evaluate patient progress during a given session and provide, on the given modality during the session, at least one of:
increased therapeutic challenge on the modality; and
decreased therapeutic challenge on the modality.

27. The method according to claim 22, including allocating services to said first multiple multipurpose location and said second multiple multipurpose location from the group of services comprising services related to: therapists, Physical Medicine diagnostic equipment, Physical Medicine treatment equipment, neurological treatment units, computer systems, billing modules, communication networks and patient transportation.

28. A method of locating a multiple multipurpose location for treating a group of patients for a fixed period of time, the method comprising:
a) locating a first multipurpose location;
b) designating a first group of a plurality of patients located within a predetermined radius of said multipurpose location;
c) providing a plurality of rehabilitation modalities at said first multipurpose location for a first fixed period of time; and
d) treating said first group of a plurality of patients with said plurality of rehabilitation modalities for said first fixed period of time.

29. The method according to claim 28, including:
e) removing said plurality of rehabilitation modalities from said multipurpose location following said first fixed period of time.

30. The method according to claim 28, wherein said first group of said plurality of patients have similar rehabilitation needs.

31. The method according to claim 29, including:
f) repeating steps a through e in a second multipurpose location with a second group of a plurality of patients for a second fixed period of time.

32. The method according to claim 31, wherein said second group of said plurality of patients have similar rehabilitation needs.
